(19) United States
(12) Patent Application Publication
(10) Pub. No.: US 2019/0110873 A1
(43) Pub. Date: Apr. 18, 2019

(54) MATERIALS AND METHODS FOR TREATING CONDITIONS ASSOCIATED WITH PATHOGENIC BIOFILM

(71) Applicant: Lea Sedghi, San Francisco, CA (US)

(72) Inventor: Lea Sedghi, San Francisco, CA (US)

(21) Appl. No.: 16/157,953

(22) Filed: Oct. 11, 2018

Related U.S. Application Data
(60) Provisional application No. 62/571,185, filed on Oct. 11, 2017.

(51) Int. Cl. A61C 15/04; A61K 36/53; A61K 36/234; A61K 36/82; A61Q 11/00

(52) U.S. Cl. CPC A61C 15/041; A61K 36/53; A61K 2035/115; A61K 36/82; A61Q 11/00; A61K 36/234

(57) ABSTRACT

The subject invention provides materials and methods that effectively support innate immunity and/or disperse pathogenic biofilms using readily available, nontoxic, natural substances, while supporting restoration of normal microbiotic homeostasis. In one embodiment, the subject invention provides anti-biofilm compositions comprising one or more prebiotic or probiotic organisms. In a further embodiment, the prebiotics are delivered via a wax disposed on a dental floss.

### CROSS REFERENCE TO RELATED APPLICATIONS

This application claims priority to U.S. Provisional Application 62/571,185, filed Oct. 11, 2017, the entirety of which is incorporated by reference.

### FIELD OF THE INVENTION

One or more embodiments relate to materials and methods that effectively support innate immunity and/or disperse pathogenic biofilms using readily available, nontoxic, natural substances, while supporting restoration of normal homeostasis microbiome of the dental surfaces and supporting surfaces.

### BACKGROUND OF THE INVENTION

The utilization of pre- and probiotics to restore and maintain dental health has been previously recognized and is in itself an established concept. However, current mechanisms of delivery disregard the importance of accurate delivery and continued maintenance of pre- and probiotics to areas between the dental surfaces that are difficult to reach. Moreover, many pre- and probiotic products neglect to acknowledge the unique nature of the oral cavity. The mouth is a highly heterogeneous and dynamic environment and harbors distinct environments with corresponding unique microbial communities. Products that seek to restore homeostasis in the oral cavity should utilize pre- and probiotics that are specified to this environment. Additionally, these substances should be applied in such a way that acknowledges the sequential colonization patterns of the dental microbiome and to support beneficial bacteria within the unique micro-niches that are created.

The dental surfaces are distinct from other environments within the body as well as other habitats within the oral cavity. Dental surfaces are unique in that they are intrinsically stagnant; an inherent shedding mechanism to alleviate accumulation of bacteria and debris does not exist at these surfaces. As a result, the dental surfaces are prone to continued bacterial colonization and the creation of complex biofilm communities. Mechanical obstruction of the dental biofilm is necessitated by the use of a toothbrush twice daily to compensate for the lack of a natural cleansing mechanism. However, the use of mechanical obstruction intermittently between brushing periods to persistently abate accumulation of accruing biofilm is not currently practiced. Current methods of preventative dental care fail to regard the importance of mechanical obstruction of accruing dental biofilm intermittently between brushing periods to the prevent excessive accumulation of plaque.

Microbial biofilms are often associated with many pathological states relating to various disease processes. Biofilm communities are defined as highly organized microbial communities that develop in a sequential manner upon an initial solid support. These communities are formed via properties relating to receptor recognition, physical protection mechanisms, as well as physical and metabolic interdependencies. Mixed species biofilms are highly heterogeneous. These microbial communities depend upon the individual species that compose the biofilm community, and the physical and metabolic interactions that occur among these individual species. Ultimately, microbial species residing within such microbial communities become distinct from their individual counterparts cultured in isolation. The establishment of a biofilm community is a step-wise and sequential process in which the colonization of one microbe is dependent upon the previous colonization of another microbe either via physical or metabolic pre-requisites and interdependencies. Micro-niches are created within the community in which the metabolic products of one species is utilized as a substrate for another, and so on. This interconnectedness of microbial metabolism within the biofilm community results in a distinct and predictable biogeography and architecture of the dental biofilm.

The excessive accumulation of biofilm communities on the dental surfaces results in the formation of dental plaque. Dental plaque exists as a highly complex biofilm community that is associated various states of dental disease, including but not limited to dental caries and periodontitis. Dental caries is associated with the destruction of the dental enamel caused by demineralization of dental enamel by acidogenic species. Periodontal disease is alternatively caused by a host immune response to the presence of excessive dental plaque and results in the destruction of the supporting structures of the tooth. Both dental caries and periodontal disease are incited and driven largely by microbial dysbiosis. Microbial dysbiosis is defined as changes in microbial community dynamics that disrupt normal homeostasis among biofilm communities. Dysbiosis encourages the proliferation of opportunistic bacteria having pathogenic potential and decreases the relative abundance of commensal species. Excessive consumption of dietary carbohydrates is additionally associated with dysbiotic communities, as these dietary-derived carbohydrates provide excess substrates for acidogenic bacteria. This results in increased production of acidic metabolic products and the creation of an acidic environment that many commensal species are unable to tolerate.

Increased microbial population diversity is associated with increased community stability and resiliency to changing environmental challenges. Population diversity is characterized by the distribution of microbial species within the community in terms of species richness and evenness. Communities in which specific species become over or under represented compared to homeostatic conditions are characteristic of dysbiotic states. The pathogenicity of the dental biofilm is directly related to the representation of species residing within the biofilm, thus determining the overall metabolic activity of the biofilm. The composition of oral biofilm communities differentiates states of oral health from states of dental disease and pathology.

The human microbiota contains more than 750 species of oral bacteria maintained within biofilms. Currently, the etiological causes of dental caries and periodontal disease are attributed largely to individual microbial species such as Streptococcus mutans and Porphyromonas gingivalis, respectively. The investigation of the etiological causes of dental disease from an individualistic approach has hindered the understanding of the polymicrobial processes leading to dental disease. To attain a true understanding of and to properly address dental disease, it is necessary to study the processes that cause dental disease progression from healthy oral microbial communities utilizing a polymicrobial perspective and systemics microbiology approach. Rather than attribution to single bacterial species, dental disease can be attributed to dysbiosis among polymicrobial interactions and thus be defined as multifactorial in nature.

States of microbial homeostasis may be attained through use of oral pre- and probiotics that seek to establish healthy biofilm communities that promote the initial colonization of commensal microbial species and the subsequent maintenance of healthy oral biofilm communities overall. Current methods of dental disease treatment and prevention focus largely on treatment and interception of existing dental disease and neglect the need for noninvasive methods of dental disease prevention that strive predominantly to restore microbial homeostasis. Effective prevention of microbial dysbiosis and subsequent dental disease should instead focus on attaining states of microbial community homeostasis and encouraging the establishment of commensal microbial species. Probiotics are recognized as living microbial organisms that are directly administered to the patient in an attempt to introduce healthy microbial species to an environment. Prebiotics are non-digestible substances that impact existing microbial communities by supporting proliferation and viability of specific species. Prebiotics subsequently induce probiotic effects in that they support the growth of indigenous commensal bacteria associated with states of health.

The effects of pre- and probiotics seek to alter the composition and enzymatic activity of the microbiome in such a way that benefits the host. Probiotics infer a beneficial effect via many mechanisms, including but not limited to competitive inhibition of pathogenic microbes, modulation of the host immune response by reducing production of pro-inflammatory cytokines, as well as inhibiting the growth of pathogenic species by producing metabolic by-products that inhibit or are antagonistic to their growth. The effect of prebiotics is more or less indirect, as prebiotics encourage the growth of beneficial members of the existing microbial community rather than directly introduce beneficial species of bacteria.

Initial colonization of the clean dental surfaces and salivary pellicle coat ensues within five minutes following brushing. Initial colonization of the dental enamel surface is succeeded by subsequent colonization. Continued colonization of the dental surface by microbes ultimately creates complex biofilm communities, colloquially known as dental plaque. Plaque is composed of various elements including but not limited to various microbes and their metabolic products, various salivary substrates, and food debris. Undisturbed, dental plaque becomes calcified and becomes dental calculus within three days of initial plaque formation. Calculus acts as a catalyst for dental disease in that it harbors pathogenic oral bacteria and retains their presence among the gingival tissue. Dental plaque is the primary cause of many states of dental disease, including periodontitis and gingivitis.

The dental surface in unique in that it lacks a natural shedding mechanism that aids in disrupting the formation of microbial biofilm communities. Many animal studies have focused on the importance of dietary texture to the development of zoological dental disease. These studies have observed the variances in oral health among animals living in the wild compared to those living in captivity. Animal studies focused on the importance of dietary texture to oral health have also observed the significance of providing fibrous dental chews to domestic animals to reduce dental plaque accumulation and relieve dental disease. Studies that have investigated the effect of soft diets on the oral health of animals in captivity and among domestic pets suggest a negative correlation between increased incidence of existing and future dental disease and dietary texture.

The addition of textured components to animal diets within these animal studies demonstrates the importance of dietary texture to both relieving existing dental disease as well as preventing future disease. While mechanical obstruction of dental plaque is necessitated by means of a toothbrush in humans, the utilization of dietary texture intermittent between brushing periods to continually act as an abrasive component on the dental surfaces is not currently practiced. This invention utilizes insoluble fiber components of non-digestible carbohydrates (cellulose, hemicellulose, and lignin) as a fibrous mechanical challenge to both aid in dispersion of existing dental biofilm as well as prevent plaque accumulation. Textured components are provided in toothpaste to aid in obstruction of preexisting plaque accumulation and are also included within a chewing gum product to aid in preventing plaque accumulation intermittent between brushing periods.

### SUMMARY OF THE INVENTION

This invention directly addresses the current need for non-invasive anti-biofilm treatments. One or more embodiments relate to materials and methods that effectively support innate immunity and/or disperse pathogenic biofilms using readily available, nontoxic, natural substances, while supporting restoration of normal microbiotic homeostasis.

The compositions of the subject invention can be delivered to the affected tissues by direct application utilizing current dental practices, thus maximizing both efficacy and safety. Examples of such current dental practices currently in widespread use include floss, toothpaste, and mouthwash. In each case, the composition of the present invention is designed to be delivered via a physical vehicle designed to be physiologically appropriate to effectively deliver targeted treatments to disperse pathogenic biofilms.

In a further embodiment of the present invention, the restoration of normal microbial homeostasis is achieved by a targeted delivery of prebiotics delivered in combination with floss, toothpaste, and mouthwash. Further embodiments of the present invention may use a two or more-step application process, e.g., localized application of a first composition via floss to decrease pathological biofilms, followed by application of a second composition via mouthwash to promote restoration of normal commensal bacterial homeostasis. In a further embodiment, the prebiotics and probiotics are delivered via a wax disposed on a dental floss.

Additional features and advantages will be set forth in the description which follows, and in part will be obvious from the description, or may be learned by the practice of such exemplary embodiments. The features and advantages of such embodiments may be realized and obtained by means of the instruments and combinations particularly pointed out in the appended claims. These and other features will become more fully apparent from the following description and appended claims, or may be learned by the practice of such exemplary embodiments as set forth hereinafter.

### BRIEF DESCRIPTION OF THE DRAWINGS

In order to describe the manner in which the above recited and other advantages and features of one or more embodiments can be obtained, a more particular description will be rendered by reference to specific embodiments thereof that are illustrated in the accompanying drawings. These drawings depict only typical embodiments, and are not therefore considered to be limiting of its scope. Accordingly, various embodiments will be described and explained with additional specificity and detail using the accompanying drawings.

FIG. 1 is a flow diagram of the general process according to an embodiment.

FIG. 2 is a flow diagram of the “Clear” step according to an embodiment.

FIG. 3 is a flow diagram of the “Promote” step according to an embodiment.

FIG. 4 is a flow diagram of the “Prevent” step according to an embodiment.

### DETAILED DESCRIPTION

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention.

This invention directly addresses the current need for non-invasive anti-biofilm treatments. One or more embodiments relate to materials and methods that effectively support innate immunity and/or disperse pathogenic biofilms using readily available, nontoxic, natural substances, while supporting restoration of normal microbiotic homeostasis.

The compositions of the subject invention can be delivered to the affected tissues by direct application utilizing current dental practices, thus maximizing both efficacy and safety. Examples of such current dental practices currently in widespread use include floss, toothpaste, and mouthwash. In each case, the composition of the present invention is designed to be delivered via a physical vehicle designed to be physiologically appropriate to effectively deliver targeted treatments to disperse pathogenic biofilms.

In a further embodiment of the present invention, the restoration of normal microbiotic homeostasis is achieved by a targeted delivery of prebiotics delivered in combination with floss, toothpaste, and mouthwash. Further embodiments of the present invention may use a two or more-step application process, e.g., localized application of a first composition via floss to decrease pathological biofilms, followed by application of a second composition via mouthwash to promote restoration of normal commensal bacterial homeostasis. In a further embodiment, the prebiotics and probiotics are delivered via a wax disposed on a dental floss. In a further embodiment, the wax itself comprises prebiotic and probiotic substances.

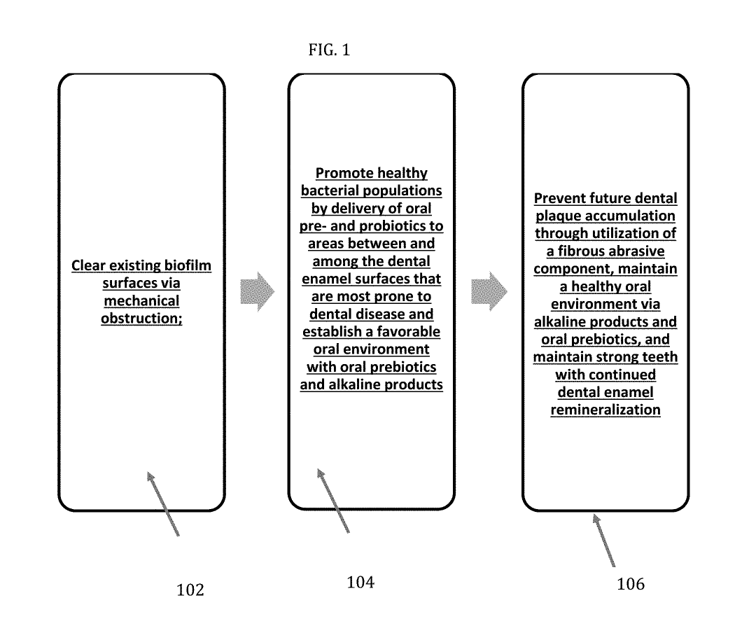

In a further embodiment, the subject invention utilizes a three-step mechanism (Clear, Promote, Prevent) to effectively restore microbial homeostasis. FIG. 1 is a flow diagram of the general process according to an embodiment wherein the 3 steps are detailed.

The first step 102, “Clear,” disrupts existing biofilm surfaces via mechanical obstruction necessitated by use of a toothbrush as well as textured components such as lignin and powdered cellulose. The second step 104, “Promote”, comprises delivery of oral pre- and probiotics to areas between and among the dental enamel surfaces that are most prone to dental disease and to establish a favorable oral environment with oral prebiotics and alkaline products. The third step 106, “Prevent”, is to prevent future dental plaque accumulation through utilization of a fibrous abrasive component, maintain a healthy oral environment via alkaline products and oral prebiotics, and maintain strong teeth with continued dental enamel remineralization

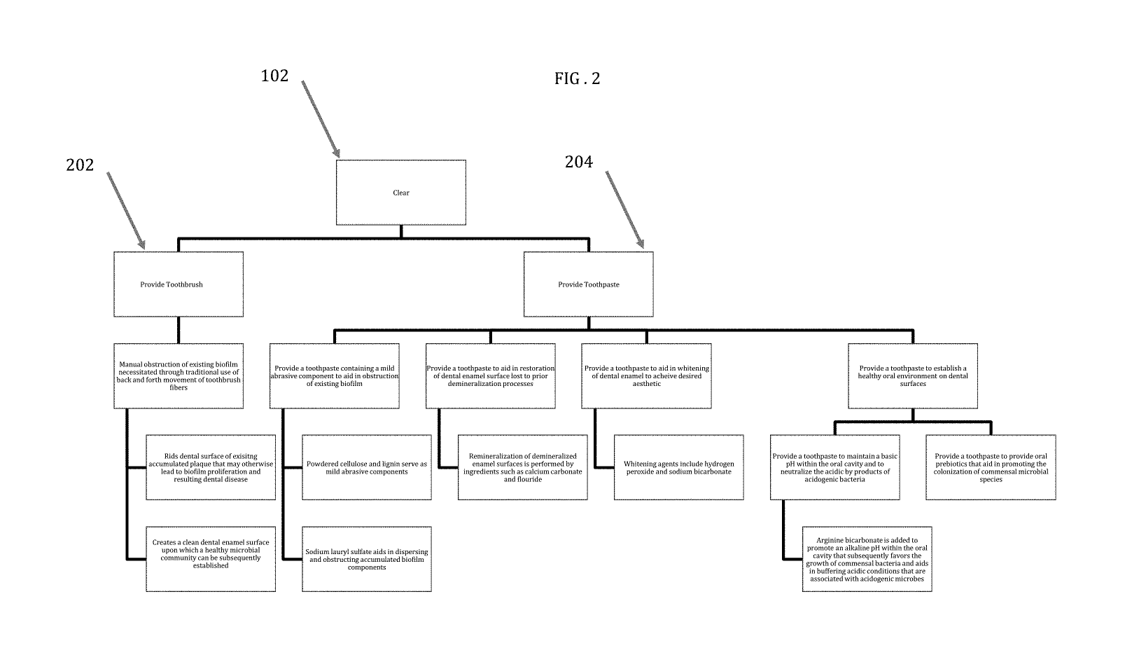

FIG. 2 is an architecture diagram of the “Clear” step according to an embodiment. The clear step includes providing toothbrush 202 and providing toothpaste 204.

The provided toothpaste associated with this step will include oral prebiotics that will aid in establishing an environment within the oral cavity that promotes the growth of commensal bacterial species. Prebiotics used within the Clear include but are not limited to plant-derived polyphenols and tannins, green tea and parsley extract, peppermint oil, and arginine bicarbonate. Initial colonization of clean dental pellicle occurs within five minutes following brushing. Thus, it is imperative that the oral environment favors the growth of beneficial primary colonizers that will impact subsequent growth. The Clear step will also focus on the reestablishment of a healthy enamel surface by including ingredients within the toothpaste product that act to effectively restore enamel surface lost to preexisting processes of demineralization and cavitation, such as fluoride and calcium carbonate.

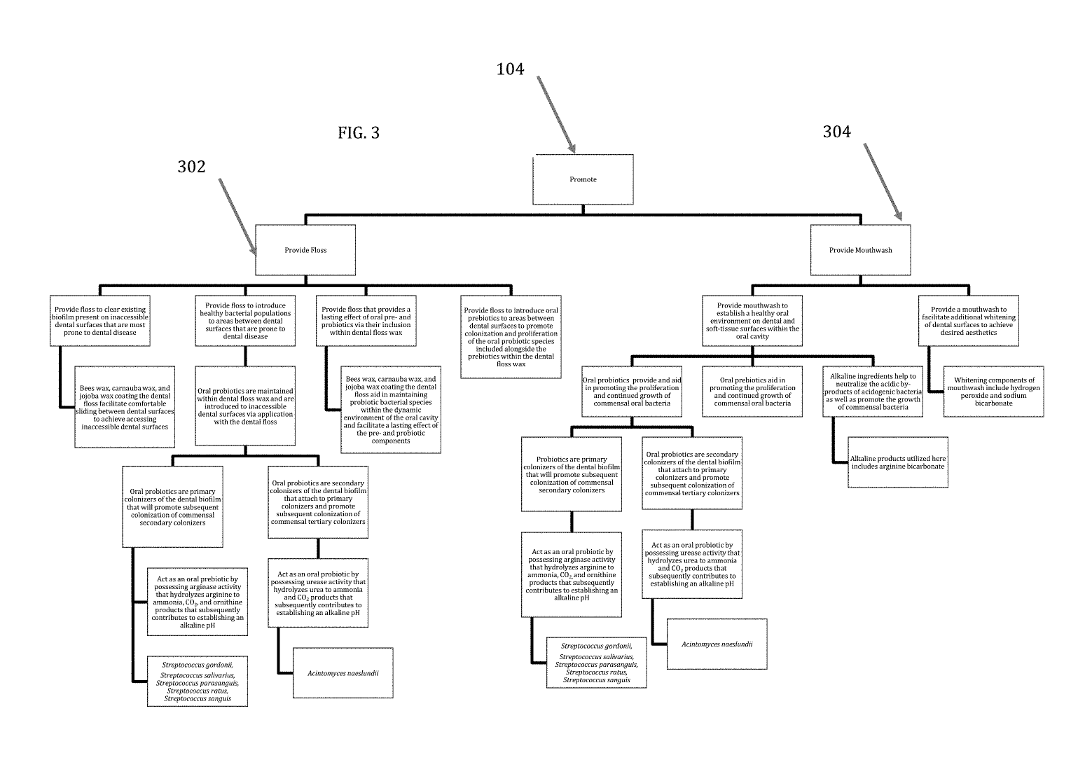

FIG. 3 is an architecture diagram of the “Promote” step according to an embodiment. The promote step includes providing floss 302 and providing mouthwash 304. The second step, “Promote,” includes the direct delivery of oral pre- and probiotics to areas between and among the dental enamel surfaces that are most prone to dental disease. In a further embodiment, the probiotics are delivered in a sequential manner to mimic natural processes of biofilm colonization. The “Promote” step utilizes primary and secondary colonizers of the oral biofilm that provide probiotic properties as well as serve as attachment sites for sequential colonization of commensal tertiary colonizers. While supragingival dental plaque is easily dislodged from smooth enamel surfaces, tight spaces between dental surfaces are not easily accessible. As a result, microbial communities tend to accrue on dental surfaces that are sheltered from traditional methods of mechanical abrasion. In one embodiment, dental floss 302 is coated with a waxy substance containing synbiotic (containing both pre- and probiotic), applied here to effectively deliver these substances as well as maintain their effects within the dynamic oral environment. Additionally, mouthwash 304 containing oral pre- and probiotics is administered to coat exposed dental enamel surfaces.

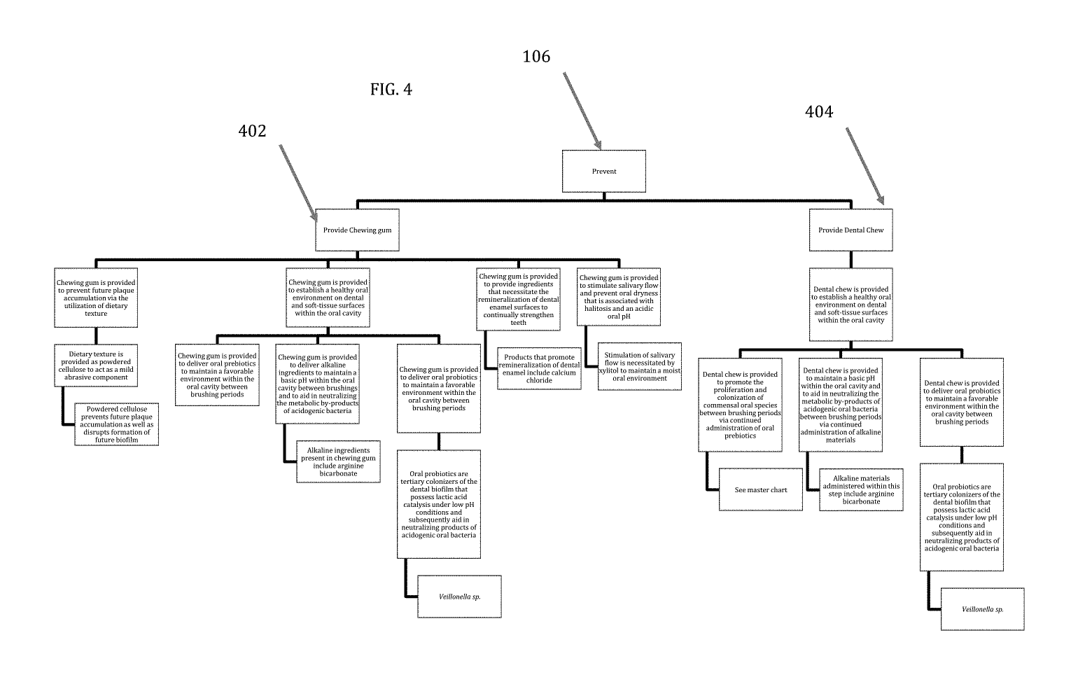

FIG. 4 is an architecture diagram of the “Prevent” step according to an embodiment. The prevent step, according to an embodiment, includes providing chewing gum 402 and/or providing dental chew 404. The final step, “Prevent,” seeks to impede the proliferation of future dental plaque. Vehicles such as dental chew 404 and chewing gum 402 are utilized here to maintain a healthy oral environment that encourages the continued presence of commensal oral bacteria. Oral prebiotics will provide metabolic substrates that promote the growth of commensal bacteria, bacteriocins that prevent the growth of pathogenic bacteria, as well as various substances that restore a slightly alkaline pH within the oral cavity. In addition to oral prebiotics, the final prevention step also accounts for the importance of mechanical obstruction for not only ridding existing disease (as with Clear), but also to preventing future plaque accumulation. Lignin and powdered cellulose exist as a fibrous, textured components that are included in the chewing gum to effectively control buildup of biofilm among the dental surface. In further embodiment, commensal tertiary colonizers of the oral biofilm are administered as probiotic components of the “Prevent” step to aid in the neutralization of the acidic by-products of acidogenic bacterial species between brushing periods.

Some ingredients common to many, but not all, embodiments of the compositions of said prebiotics include xylitol, fluoride, zinc, copper, nickel, selenium, strontium, polyphenols, phenolic acids, flavonoids, oleanoic acid, oleanoic aldehyde, liniolic acid, betulin, betulinic acid, 5-(hydroxymethyl)-2-furfural, rutin, beta-sitosterol, beta-sitosterol glucoside, bisflavanols, theaflavins, theaflagallins, epiflavic acid, thearubigins, epigallocatechin, epicatechin, gallocatechin, (+) catechins, high MW cranberry polyphenols, cocoa phenol pentamers, cocoa bean husk, trigonelline, caffeine, chlorogenic acid, apple polyphenols, grape seed extract (proanthrocyanidins), propolis (sealant), sodium benzoate, potassium sorbate, sodium nitrite, phosphate, calcium, casein, lactoferrin, lysozyme, lactoperoxidase, folate-binding protein, immunoglobins, proteose peptones, transferrin, epidermal growth factor, tissue growth factor (beta), insulin-like growth factor, plasmin, xanthine oxidase, glucose oxidase, arginine, CPP-ACP, Novamin, tricalcium phosphate, nanohydroxyapetite, dicalcium phosphate dehydrate, CPP-ACP, CPP-ACFP, CD-CP, proanthrocyanidins, arabinose, xylose, and Hydrogen peroxide. Other ingredients that may be used in certain embodiments include, but are not limited to, prebiotic compounds such as larch or acacia gum, other hive products such as royal jelly, bee bread and propolis, green tea derivatives such as epigallocatechin gallate (EGCG) and L-theanine, other plant derivatives such as from Inula helenium, Aletaleuca alternifolia and Leptosperrnum scoparium and water-soluble and water-insoluble Vitamin D3.

Another advantage of the current invention is the established safety of its components. The compositions described herein are composed of components that have already been individually established to be safe. In preferred embodiments, the compositions of the subject invention comprise a mixture of natural, generally regarded as safe (GRAS) ingredients.

The purpose of the “Clear” step is to clear away existing dental biofilm. Mechanical obstruction of existing biofilm will be performed by the existing method necessitated by a toothbrush. The Clear step also employs a specialized toothpaste 204 to aid in the obstruction of existing biofilm. The purpose of the toothpaste is to clear away existing biofilm to establish a clean dental enamel surfaces using mild abrasive action provided by added fibrous components (hemicellulose, cellulose, lignin) and known dispersants (sodium lauryl sulfate). Additionally, restoration of lost dental enamel and strengthening of existing enamel will be employed using known substances that aid in rebuilding and strengthening the enamel surface (calcium carbonate, calcium chloride, fluoride). A healthy oral environment will be promoted using bacteriocins (nisin) and prebiotics with known benefit to the oral cavity (peppermint oil, parsley extract, green tea extract, eucalyptus oil). Such prebiotics are strategically applied in this primary step to establish a favorable oral environment in which subsequent probiotics characteristic of early biofilm formation will be applied. The prebiotics will also induce a slightly alkaline environment to aid in restoration of optimal oral pH as well as help to maintain a higher pH during acidic challenge to the oral cavity via metabolism of acidogenic species and/or consumption of acidic foods (arginine bicarbonate). Desired dental aesthetics will be achieved by use of known whitening substances (hydrogen peroxide, sodium bicarbonate). A desired feeling of oral cleanliness and freshness will be achieved via substances that aid in moistening of the oral cavity (glycerin, water, xylitol) and abatement of halitosis (sodium bicarbonate, peppermint oil, zinc citrate). Additional substances will be used to deliver desired product flavor (peppermint oil, xylitol) as well as provide appropriate product consistency (water, carrageenan). Toothpaste 204 ingredients may comprise one or more of the following ingredients:
Calcium carbonate (mild abrasive, enamel remineralization)
Cellulose, hemicellulose, lignin (mild abrasive)
Calcium chloride (enamel remineralization)
Fluoride (enamel remineralization)
Xylitol (flavor component, stimulates salivary flow)
Hydrated silica (product consistency)
Nisin (bacterocin)
Zinc citrate (eliminates halitosis)
Peppermint oil (eliminates halitosis, flavor component, prebiotic component)
Sodium bicarbonate (eliminates halitosis)
Hydrogen peroxide (whitening agent)
Sodium bicarbonate (whitening agent)
Arginine bicarbonate (promotes alkaline pH)
Eucalyptus oil (prebiotic component)
Green tea extract (prebiotic component)
Parsley extract (prebiotic component)
Glycerin (oral cavity moistener)
Water (product consistency)
Sodium lauryl sulfate (dispersant of oral biofilm)
Carrageenan (product thickener)

The purpose of the “Promote” step is to aid in clearance of existing biofilm present on dental surfaces that are largely inaccessible or difficult to reach. Such clearance will be necessitated by dental floss. Flossing away existing biofilm will occur simultaneously with the delivery of oral pre- and probiotics to these sites. In one embodiment, oral pre- and probiotics are delivered to such sites via a wax coating the dental floss 302. The wax serves to both retain the pre- and probiotics and to deliver and maintain the presence of such substances in hard to reach areas (bees wax, carnauba wax, jojoba wax). Bacteriocins (nisin) and prebiotics within the wax (eucalyptus oil, green tea extract, parsley extract) act to provide a healthy environment upon which subsequent probiotics will be introduced and future colonization will occur. Probiotics utilized are bacteria with known relationship to the oral cavity that provide benefit via neutralization of acidic by-products of acidogenic species and via production of alkaline substances. Bacteria utilizing the arginase deaminase system to catabolize arginine to ornithine, ammonia, and CO₂ will be utilized (Streptococcus salivarius, Streptococcus gordonii, Streptococcus parasanguinis, Streptococcus intermedius, Streptococcus ratus, Streptococcus sanguinis). Bacteria utilizing urease activity to produce ammonia and CO₂ via urea catabolism will additionally by utilized (Actinomyces naeslundii), as well as bacteria that demonstrate low pKa lactic acid catalysis to neutralize products of acidogenic bacteria (Veillonella sp.). Product flavor components (acacia senegal gum, spearmint), halitosis-eliminating components (propolis extract, commiphora myrrha, spearmint, and substances that promote lubrication of the oral cavity (glycerin, sorbitol) are also included.

The “Promote” step also seeks to create a favorable oral environment upon readily accessible sites including the dental and supporting soft tissue structures by application of oral bacteriocins (nisin), prebiotics (eucalyptus oil, green tea extract, parsley extract), and probiotics (listed above) via a mouthwash that provides a broad application to many surfaces within the oral cavity. Substances that encourage alkaline pH (arginine bicarbonate) will also be utilized. The promote step also seeks to eliminate halitosis (peppermint oil, spearmint oil, zinc chloride, menthol). Whitening (hydrogen peroxide, sodium bicarbonate) and re-mineralizing substances of dental enamel (calcium carbonate, calcium chloride, fluoride) will also be performed at this step. Substances related to product consistency (propanediol) and flavor (xylitol) are also included. The floss may comprise of one or more of the following ingredients. Ingredients:
Bees wax (facilitates comfortable sliding between dental surfaces, retains oral pre- and probiotics)
Carnauba wax (facilitates comfortable sliding between dental surfaces, retains oral pre- and probiotics)
Spearmint (product flavor, eliminates halitosis, prebiotic component)
Jojoba wax (facilitates comfortable sliding between dental surfaces, retains oral pre- and probiotics)
Propolis extract (eliminates halitosis)
Commiphora myrrha resin extract (eliminates halitosis)
Nisin (bacteriocin)
Glycerin (lubricates oral cavity)
Sorbitol (lubricates oral cavity)
Eucalyptus oil (prebiotic component)
Green tea extract (prebiotic component)
Parsley extract (prebiotic component)
Streptococcus salivarius (production of antibiotic bacteriocins, modulation and reduction of host inflammatory response, antagonist of virulent Streptococci involved in tooth decay and periodontitis via production of bacteriocins, ADS activity to restore alkaline pH via catabolism of arginine to ornithine, ammonia, and CO₂).
Streptococcus gordonii (ADS activity to restore alkaline pH via catabolism of arginine to ornithine, ammonia, and CO₂).
Streptococcus parasinguinis (ADS activity to restore alkaline pH via catabolism of arginine to ornithine, ammonia, and CO₂).
Streptococcus intermedius (ADS activity to restore alkali pH conditions via arginine catabolism to release ornithine, ammonia, and CO₂).
Streptococcus ratus (ADS activity to restore alkali pH conditions via arginine catabolism to release ornithine, ammonia, and CO₂).
Streptococcus sanguinis (ADS activity to restore alkali pH conditions via arginine catabolism to release ornithine, ammonia, and CO₂).
Veillonella sp. (Low pKa lactic acid catalysis—aids in neutralizing products of acidogenic oral bacteria)
Actinomyces naeslundii (urease activity to restore alkali pH conditions via hydrolysis of urea to ammonia in CO₂).

The purpose of the “Prevent” step is to aid in prevention of future dental plaque as well as establish a healthy oral environment. Chewing gum 402 will aid in preventing the accumulation of future biofilm via the utilization of fibrous components (hemicellulose, cellulose, lignin) that act as a mild abrasive component to the dental surfaces. Additionally, the fibrous texture of the gum 402 will act to stimulate the salivary glands to increase saliva production that provides inherent anti-bacterial action. The “Prevent” step also seeks to establish a healthy oral environment via the introduction of oral bacteriocins (nisin) and prebiotics (peppermint oil, spearmint oil, green tea extract, eucalyptus oil), as well as substances that promote alkalinity (arginine bicarbonate). This step also aids in elimination of halitosis (peppermint oil, spearmint oil, green tea extract). Continuous remineralization of the dental enamel surfaces will also be provided in this step by mild substances that act to remineralize lost enamel (calcium chloride). Substances relating to product flavor (peppermint oil, spearmint oil, menthol, xylitol), and consistency (gum base, gum arabic) are also included.

The “Prevent” step also includes dental chews 404 that seek to maintain a healthy oral environment via bacteriocins (nisin) and prebiotic substances (green tea extract, parsley extract, peppermint oil) and substances that promote alkalinity within the oral cavity (arginine bicarbonate). Substances that provide remineralization and strengthening of dental enamel are also included (calcium carbonate, calcium chloride, fluoride). Probiotic bacteria with known relationship to the oral cavity that provide benefit via neutralization of acidic by-products of acidogenic species and by production of alkaline substances are also included. Bacteria utilizing the arginase deaminase system to catabolize arginine to ornithine, ammonia, and CO₂ will be utilized (Streptococcus salivarius, Streptococcus gordonii, Streptococcus parasanguinis, Streptococcus intermedius, Streptococcus ratus, Streptococcus sanguinis). Bacteria utilizing urease activity to produce ammonia and CO₂ via urea catabolism will additionally by utilized (Actinomyces naeslundii), as well as bacteria that demonstrate low pKa lactic acid catalysis to neutralize products of acidogenic bacteria (Veillonella sp.). Substances relating to product consistency (soy lechitin, diglycerides and monoglycerides) and flavor (xylitol, peppermint oil) and lubrication of the oral cavity (palm oil) are also included.

The chewing gum may have one or more of the following ingredients. Ingredients:
Xylitol (product flavor, stimulates salivary glands)
Gum base (water-insoluble masticatory delivery system)
Peppermint oil (product flavor, prebiotic component, eliminates halitosis)
Spearment oil (product flavor, prebiotic component, eliminates halitosis)
Green tea extract (prebiotic component)
Menthol (product flavor)
Nisin (bacterocin)
Eucalyptus oil (prebiotic component)
Cellulose, hemicellulose, lignin (fibrous abrasive component)
Calcium chloride (remineralize enamel surface)
Arginine bicarbonate (promotes alkaline pH)
Gum Arabic (emulsifier)

The dental chews may have one or more of the following ingredients. Ingredients:
Calcium carbonate (remineralizes enamel surface)
Calcium chloride (remineralizes enamel surface)
Fluoride (remineralizes enamel surfaces)
Palm oil (lubricates enamel surface)
Arginine bicarbonate (alkaline product)
Xylitol (flavor component, stimulates salivary glands)
Nisin (bacterocin)
Eucalyptus oil (prebiotic component)
Green tea extract (prebiotic component)
Parsley extract (prebiotic component)
Peppermint oil (product flavor, eliminates halitosis)
Menthol (product flavor, eliminates halitosis)
Streptococcus salivarius (production of antibiotic bacteriocins, modulation and reduction of host inflammatory response, antagonist of virulent Streptococci involved in tooth decay and periodontitis via production of bacteriocins, ADS activity to restore alkaline pH via catabolism of arginine to ornithine, ammonia, and CO₂).
Streptococcus gordonii (ADS activity to restore alkaline pH via catabolism of arginine to ornithine, ammonia, and CO₂).
Streptococcus parasinguinis (ADS activity to restore alkaline pH via catabolism of arginine to ornithine, ammonia, and CO₂).
Streptococcus intermedius (ADS activity to restore alkali pH conditions via arginine catabolism to release ornithine, ammonia, and CO₂).
Streptococcus ratus (ADS activity to restore alkali pH conditions via arginine catabolism to release ornithine, ammonia, and CO₂).
Streptococcus sanguinis (ADS activity to restore alkali pH conditions via arginine catabolism to release ornithine, ammonia, and CO₂).
Veillonella sp. (Low pKa lactic acid catalysis—aids in neutralizing products of acidogenic oral bacteria)
Actinomyces naeslundii (urease activity to restore alkali pH conditions via hydrolysis of urea to ammonia in CO₂).
Soy Lechithin (product emulsifier)
Diglycerides and monoglycerides (product emulsifier)

1. A method for treating conditions associated with pathogenic biofilm, the method comprising:
clearing existing biofilm surfaces via mechanical obstruction, wherein the mechanical obstruction further comprises use of a toothbrush in combination with a toothpaste containing textured components such as lignin and powdered cellulose;
promoting healthy bacterial populations by delivery of oral prebiotics and probiotics to areas between and among the dental enamel surfaces that are most prone to dental disease; and
preventing the proliferation of future dental plaque by intermittently utilizing a prevention vehicle.

2. The method of claim 1, wherein the promoting healthy bacterial populations by delivery of oral pre- and probiotics further comprises applying dental floss coated with a waxy substance containing both pre- and probiotics

3. The method of claim 2, wherein the prevention step further comprises intermittently utilizing dental chews to maintain a healthy oral environment that encourages the continued presence of commensal oral bacteria.

4. The method of claim 3, wherein the dental chews are administered intermittently between brushing periods to lessen accumulation of accruing biofilm and to deliver oral pre- and probiotics.

5. The method of claim 4, wherein the dental chews further comprise green tea extract, Streptococcus gordonii, and Veillonella sp.

6. The method of claim 5, wherein the probiotics are delivered in a sequential manner to mimic the natural processes of biofilm colonization.

7. The method of claim 6, further comprising utilizing primary and secondary colonizers of the oral biofilm that provide probiotic properties as well as serve as attachment sites for sequential colonization of commensal tertiary colonizers.

8. The method of claim 2, wherein the waxy substance of the dental floss is a prebiotic.

9. The method of claim 8, wherein the waxy substance further comprises green tea extract and hemp fiber.

10. The method of claim 10, wherein the toothpaste further comprises oral prebiotics that will aid in establishing an environment within the oral cavity that promotes the growth of commensal bacterial species.

11. The method of claim 11, wherein the toothpaste further comprises plant-derived polyphenols and tannins, green tea and parsley extract, peppermint oil, and arginine bicarbonate.
